(19) United States
(12) Patent Application Publication
(10) Pub. No.: US 2020/0234796 A1
(43) Pub. Date: Jul. 23, 2020

(54) Evaluating Optimality Of A Trace Generated During Sequence Alignment

(71) Applicant: THE REGENTS OF THE UNIVERSITY OF MICHIGAN, Ann Arbor, MI (US)

(72) Inventors: Satish NARAYANASAMY, Ann Arbor, MI (US); Daichi FUJIKI, Ann Arbor, MI (US); David T. BLAAUW, Ann Arbor, MI (US); Reetuparna DAS, Ann Arbor, MI (US); Shunhao WU, Ann Arbor, MI (US)

(21) Appl. No.: 16/749,679

(22) Filed: Jan. 22, 2020

Related U.S. Application Data
(60) Provisional application No. 62/795,202, filed on Jan. 22, 2019.

(51) Int. Cl. G16B 30/10

(52) U.S. Cl. CPC G16B 30/10

(57) ABSTRACT

A method is presented for aligning a read with a reference substring of a genome sequence. The method includes: receiving a banded portion of a matrix from a sequence alignment algorithm; calculating a score threshold for the banded portion of the matrix, where value of the score threshold is calculated as a function of a scoring method used by the sequence alignment algorithm; identifying a high score amongst the cells in the banded portion of the matrix; and comparing the high score to the score threshold. Performing variant calling using the banded portion of the matrix when the high score is greater than to the score threshold. Computing alignment scores for a larger portion of the matrix using the sequence alignment algorithm when the high score is less than or equal to the score threshold.

### CROSS-REFERENCE TO RELATED APPLICATIONS

This application claims the benefit of U.S. Provisional Application No. 62/795,202, filed on Jan. 22, 2019. The entire disclosure of the above application is incorporated herein by reference.

### FIELD

The present disclosure relates to evaluating optimality of a trace generated during sequence alignment.

### BACKGROUND

Read alignment is one of the time-consuming steps in genome sequencing, which takes around 300 CPU hours with the state-of-the-art software running on a server with Intel's latest Xeon processors. This process is responsible for determining the candidate position of a read in the reference genome. Due to variants and sequencing errors, a read (referred as query Q) may not perfectly match a substring in the reference genome. Sequence aligners solve this problem in two steps: seeding and seed-extension. Seeding finds perfect matches in the reference genome for small substrings (seeds) in a read. The seed positions are then extended to determine the best position by using an approximate string matching algorithm based on a dynamic programming algorithm. The most widely used dynamic programming algorithms particularly designed for genomic seed extension are Smith-Waterman and Needleman-Wunsch sequence alignment algorithms. These algorithms compute similarity score between two strings by filling a matrix of size N², where N is the string length.

Matches are scored using an affine gap function, which is based on edit distance but weighs different edit types differently. The hit position for a read that yields the highest score is chosen as that read's mapping position. The final output also contains a trace of edits to the reference string needed to align the read at the chosen reference position. This final step is referred to as traceback, which constructs the trace with the optimal alignment by tracing back pointers starting from the highest scoring cell.

Smith-Waterman algorithm is used for local alignment as seen in FIG. 1A. In the local alignment, gaps at both of the ends of the read are not penalized, and the result trace is given based on the maximum score obtained by aligning a substring of the read. On the other hand, Needleman-Wunsch algorithm penalizes gaps at the ends and produces an end-to-end alignment as seen in FIG. 1C. This is called global alignment. Generally, the global alignment is considered to provide better alignment, while the local alignment is mainly used to choose candidates for the final alignment calculated by Needleman-Wunsch. Furthermore, there exist an approach called semi-global alignment, where the algorithm penalizes only gaps at one end and does not penalize the other end as seen in FIG. 1B. Considering the fact that the global alignment is employed in many alignment approaches, reducing redundant calculation in the global alignment is a key determinant for the performance regardless of hardware or software execution.

Several approaches have been explored for limiting the cells in the grid to fill by the global alignment algorithms, including fixed/adaptive banding and search based pruning. For example, fixed banding calculates scores in cells on 2K+1 band along the main diagonal running from upper-left to lower-right. These banding based approaches offers efficiency because the computational complexity is reduced to O(NK), however, they have difficulty in guaranteeing optimality of the generated alignment. Other approaches use classic best-first search algorithms, such as the A* search algorithm. With an admissible heuristic that estimates cost of the optimal path to the end, A* is guaranteed to return the optimal path with minimal exploration space where the optimality can be inferred by the heuristic. However, the cost to implement A* search is prohibitively high in both hardware and software due to the complexity of priority queues and the difficulty in parallelization.

This section provides background information related to the present disclosure which is not necessarily prior art.

### SUMMARY

This section provides a general summary of the disclosure, and is not a comprehensive disclosure of its full scope or all of its features.

In one aspect, a method is presented for aligning an unknown string with a reference string. The method includes: receiving a banded portion of a matrix from a sequence alignment algorithm; calculating a score threshold for the banded portion of the matrix, where value of the score threshold is calculated as a function of a scoring method used by the sequence alignment algorithm; identifying a high score amongst the cells in the banded portion of the matrix; and comparing the high score to the score threshold. When the high score is greater than the score threshold, variant calling is performed using the banded portion of the matrix. When the high score is less than or equal to the score threshold, alignment scores are recomputed for the entire matrix using the sequence alignment algorithm.

In another aspect, additional checking can be performed to reduce the number of reruns. In a similar manner, the method includes: receiving a first banded portion of a matrix from a sequence alignment algorithm; identifying a high score amongst the cells in the banded portion of the matrix; calculating a first score threshold for the first banded portion of the matrix, where value of the first score threshold is calculated as a function of a scoring method used by the sequence alignment algorithm; and comparing the high score to the first score threshold. The method further includes: calculating a second score threshold for the banded portion of the matrix, where value of the second score threshold is larger than the first score threshold and is calculated as a function of a scoring method used by the sequence alignment algorithm; and comparing the high score to the second score threshold. When the high score is greater than to the first score threshold and the high score is greater than the second score threshold, variant calling is performed using the banded portion of the matrix.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1A is a diagram depicting a local alignment;

FIG. 1B is a diagram depicting a semi-global alignment;

FIG. 1C is a diagram depicting a global alignment;

FIG. 2 is a flowchart illustrating a proposed method for aligning a read with a reference substring of a genome sequence;

FIG. 3 illustrates how to determine the scoring threshold from a banded portion of a matrix;

FIG. 4 is a graph showing the bandwidth in relation to coverage;

FIG. 5 is a diagram illustrating an alternative method for aligning a read with a reference substring of a genome sequence;

FIGS. 6A and 6B are example matrices showing the first score threshold and the second score threshold, respectively;

FIG. 7 is an example matrix showing an extra region for performing an edit distance check.

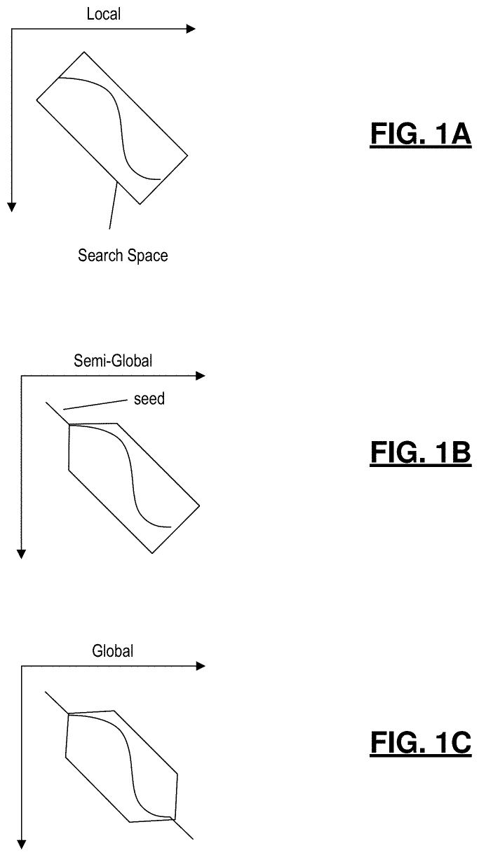

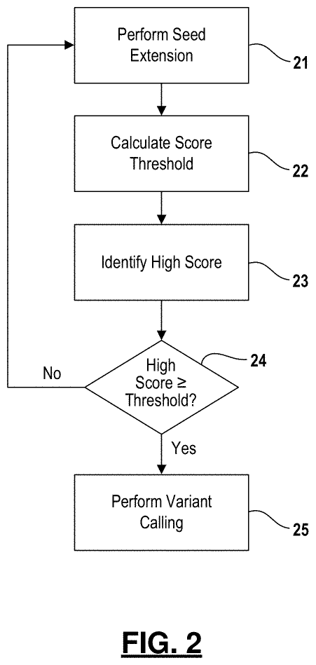

Corresponding reference numerals indicate corresponding parts throughout the several views of the drawings.

### DETAILED DESCRIPTION

Example embodiments will now be described more fully with reference to the accompanying drawings.

A genome is a long string of DNA base-pairs A, G, C, and T. Sequencers produce chopped and out-of-order reads from biological samples which are then reconstructed to form sequence whole genome. Due to variants and sequencing errors, a read (referred as query Q) may not perfectly match a substring in the reference genome. Sequence alignment algorithms are used to align reads with substrings of a reference genome.

FIG. 2 illustrates a proposed method for aligning a read with a reference substring. As a starting point, seeding and seed-extension are performed at 21 in a conventional manner. The most widely used dynamic programming algorithms particularly designed for genomic seed extension are called Smith-Waterman and Needleman-Wunsch. These algorithms compute a similarity score between two strings by filling a matrix of size N², where N is the string length. While reference is made to these two particular algorithms, it is readily understood that other types of sequence alignment algorithms fall within the scope of this disclosure.

In the example embodiment, a banded approach for sequence alignment is used. Rather that compute the entire matrix, the sequence alignment algorithm outputs a banded portion of the matrix, where each column in the matrix corresponds to a character in the given read, each row in the matrix corresponds to a character in the given reference substring, such that a value of a given cell is the alignment score between characters in the unknown string that precede and include the given cell and the characters in the reference string that precede and include the given cell. Read alignment requires the sequence of edits made and their positions in the string for the best solution found. This is called trace and can be obtained by trailing the cells with largest scores from the bottom-right cell (Needleman-Wunsch) or from the cell with maximum score (Smith-Waterman). The proposed method manifests the necessary condition of the score to grant the optimality of the trace generated within the band. In other words, the method confirms the non-existence of the optimal trace outside the region in which the band has been swept.

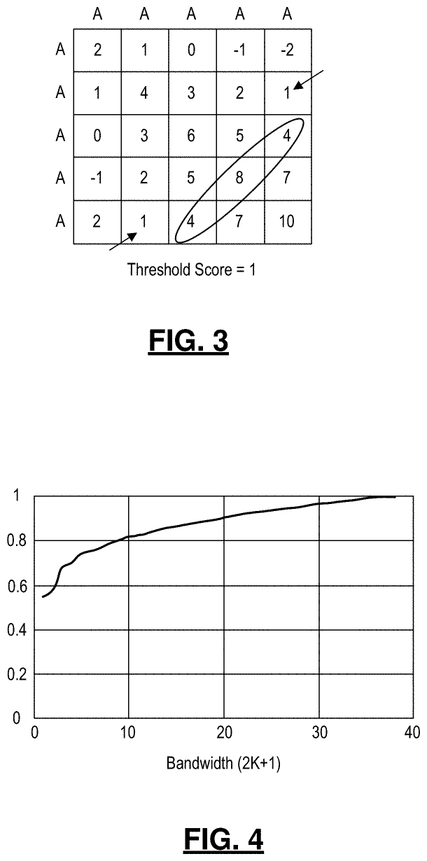

To do so, a score threshold for the banded portion of the matrix is calculated at 22. The value of the score threshold is calculated as a function of a scoring method used by the sequence alignment algorithm. More specifically, for an input pair of a read (or unknown) string and a reference string of equal length, the value of the score threshold is calculated as a function of string length N, size parameter of the banded portion of the matrix K as provided by the sequence alignment algorithm, and the scoring method used by the sequence alignment algorithm. For an input pair of different string length, the length of the longer string is used, while embodiments with an edit distance check described later enable them to use the length of the shorter string (usually the read string). In one approach, a score matrix is built assuming best alignment (i.e. all matches) and the maximum possible score for each cell is determined. For semi-global alignment, the intersections of the trajectories of the boundary cells of the larger anti-diagonal band (i.e. cells at 5,3 and 3,5 of FIG. 3) have the maximum possible score for the traces that follow the trajectories (for semi-global alignment). This score is used as the threshold. In this example, the threshold score is four. Likewise, the cells closest to the intersections but not included in the band region (i.e. cells 5,2 and 2,5 of FIG. 3) shows the maximum possible score of the trace outside the band region.

The score threshold can also be derived mathematically. In an example embodiment, the scoring method used by the sequence algorithm is further defined as a gap penalty method, such as the affine gap penalty method. For global alignment, the scoring threshold is calculated as follows:

$$s c o r {e}_{threshold} = \{ \begin{matrix}mN - ( m + 2 {g}_{o} + 2 {g}_{e} ) & if K = 0 \\ mN - ( m + 2 {g}_{o} + 2 {g}_{e} ) - 2 K ( 2 {g}_{e} + m ) & otherwise\end{matrix} .$$

where m is match reward, gₒ is gap opening penalty, gₑ is gap extension penalty, N is string length and K is size of the banded portion of the matrix. Coefficient 2 is used for gₒ and gₑ to include the gap insertion/extension needed to return to the main diagonal.

For semi-global alignment, the scoring threshold is calculated as follows:

$$scor {e}_{threshold} = \{ \begin{matrix}mN - ( m + {g}_{0} + {g}_{e} ) & if K = 0 \\ mN - ( m + {g}_{o} + {g}_{e} ) - 2 K ( 2 {g}_{e} + m ) & otherwise\end{matrix} ,$$

where m is match reward, gₒ is gap opening penalty, gₑ is gap extension penalty, N is string length and K is size of the banded portion of the matrix. Here, the maximum possible score for a cell is given by mN. For the main diagonal, the gap opening penalty, the gap extension penalty, and the match reward are subtracted from the maximum possible score; whereas, for the remainder of the band, the gap extension penalty and the match reward are subtracted. The match reward is subtracted because a diagonal lane with t gaps only compares N−t matches/mismatches.

Burrows-Wheeler Aligner (BWA) is an example software package for mapping sequences against a large reference genome and BWA-MEM is an alignment algorithm implemented by BWA. For illustration purposes, BWA-MEM uses affine gap penalty scoring SC₁ (1, 4, 6, 1) for short reads and SC₂ (1, 1, 1, 1) for long reads. Assuming a default bandwidth K=16 and a string length N=100, the score threshold is calculated as 28 [=100−(1+6+1)−2(16)(1+1)] for SC₁ and 33 for SC₂ for semi-global alignment. For K=5 hardware, the score threshold is calculated as 72 for SC₁ and 77 for SC₂. Other types of scoring methods are contemplated by this disclosure. Edit distance function is a special case of the affine gap function, where SC (1, 1, 0, 1). Assuming bandwidth K=5 and string length N=100, the scoring threshold (semi-global) is calculated as 78. From these examples, it is readily understood how to extend the calculation of the scoring threshold to other scoring methods.

Given the score threshold for the banded portion of the matrix, a determination can be made regarding the optimality of the returned trace from the sequence alignment algorithm. The high score amongst the cells in the banded portion of the matrix is identified at 23 and then compared to the score threshold at 24. When the high score is greater than the score threshold, the optimality of the returned trace is guaranteed and processing can continue to variant calling using the returned trace as indicated at 25. When the high score is less than or equal to the score threshold, the optimality of the returned trace is not guaranteed. In this case, processing returns to seed extension. Seed extension is then repeated for a larger band or with the entire matrix.

In one embodiment, the seed extension process is repeated using the entire matrix. In other embodiments, the seed extension process can be repeated one or more times using another portion of the matrix that is larger in size than the first banded portion of the matrix. Each iteration would use a banded portion of the matrix larger than the previous iteration until the result is guaranteed to be optimal or seed extension is performed using the entire matrix. The number of iterations is preferably predefined.

It is important to note that the proposed method does not alter the dynamic programming algorithm or its hardware/software implementation. Rather, logic is added to judge whether optimality can be granted with the score provided. This logic is sound but not complete. This implies following nature of the logic: (1) some results cannot be guaranteed the optimality and need to be processed by other mechanisms having full matrix or larger band, (2) some results can be categorized as “optimality cannot be guaranteed” although the trace is optimal (non-completeness), and (3) all traces guaranteed optimality by the logic are optimal (soundness).

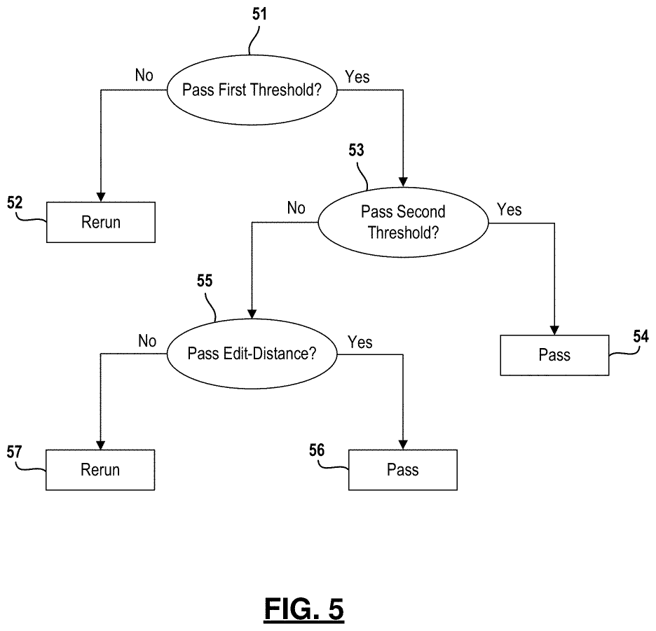

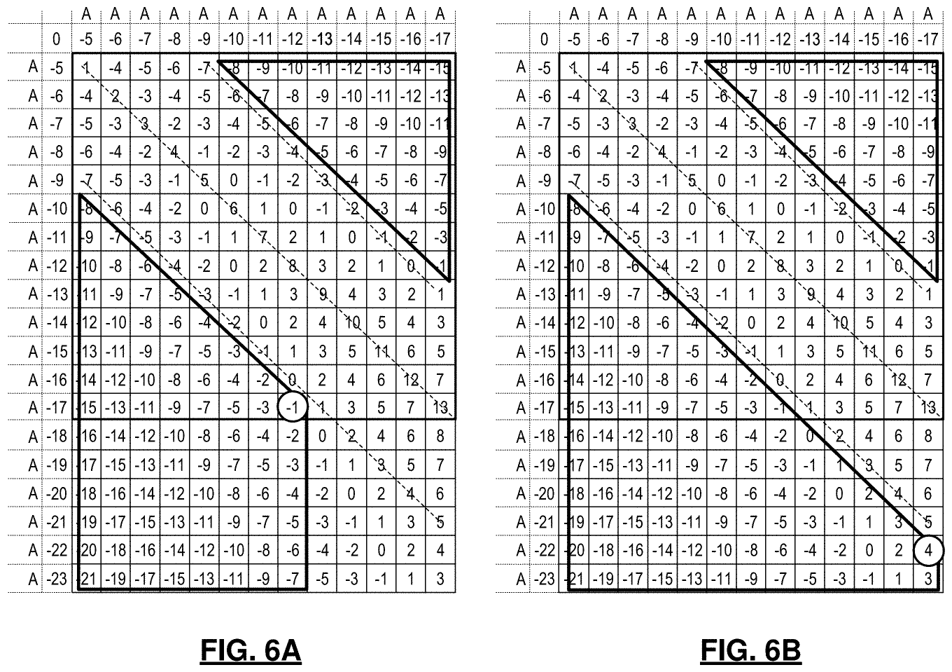

FIG. 5 depicts a variant of the method for aligning a read with a reference substring. With the thresholding mechanism described above (using bandwidth K=20), the passing rate is slightly above 70%. In order to avoid rerunning 30% of the extensions with full band, one can instead use a resource efficient edit-distance machine to perform an alignment on the end of the query string. In this alternative embodiment, the edit-distance score can be used as a check for the ones that fail the thresholding mechanism. If the narrow-band high score passes the check, there is no need to rerun the extension. Combining thresholding and the edit-distance check as will be further described below, the total passing rate for the narrow-band score is over 99%.

The alignment process begins with a first threshold condition as indicated at 51. Upon receiving a first banded portion of a matrix from a sequence alignment algorithm, a high score is identified from amongst the cells in the banded portion of the matrix and compared to a first threshold score. In one embodiment, the first threshold score is computed in the same manner as described above. If the high score is less than (or equal to) the first threshold score, alignment scores are recomputed at 52 for another portion of the matrix (e.g., the entire matrix) using the sequence alignment algorithm.

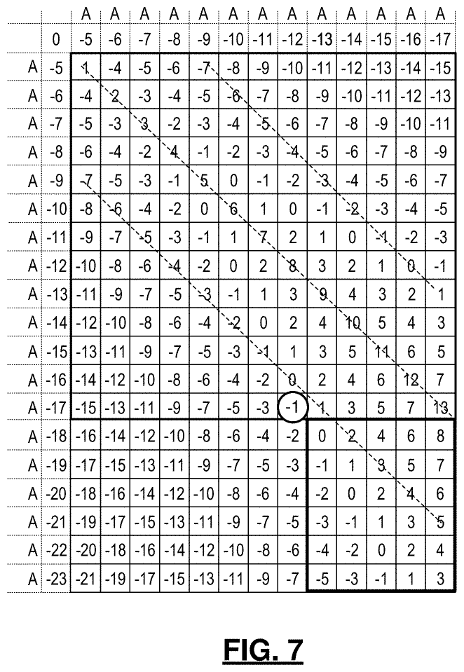

On the other hand, to reduce the number of reruns with the entire matrix, additional checking can be performed. In the example embodiment, there are two thresholds: S1 and S2, where S1 is the first threshold described above while using the smaller string length as N, and S2 will be explained later. In FIG. 6A, the first score threshold S1 (i.e., circled cell) is the largest possible score outside the band but within the black square. Passing S1 (the high score>S1) means the narrow-band score is guaranteed to be larger than any score within the highlighted region of FIG. 6A, while failing S1 (the high score<=S1) results in the recomputation of alignment score for another portion of the matrix.

With continued reference to FIG. 5, if the high score passes the first thresholding check at 51, the second thresholding check is performed at 53. To do so, the second score threshold S2 is computed, where value of the second score threshold is larger than the first score threshold and is calculated as a function of a scoring method used by the sequence alignment algorithm. For semi-global alignment, the second score threshold can be calculated using a gap penalty method as follows:

$${S}_{2} = \{ \begin{matrix}mN - ( {g}_{0} + {g}_{e} ) & if K = 0 \\ mN - ( {g}_{0} + {g}_{e} ) - 2 {Kg}_{e} & otherwise\end{matrix} .$$

where m is match reward, gₒ is gap opening penalty, gₑ is gap extension penalty, N is read length and K is size of the banded portion of the matrix. Global alignment is usually done in a perfect square matrix, but theoretically a rectangular matrix can also be used. In this case, the second score threshold S2 is derived by:

$${S}_{2} = \{ \begin{matrix}mN - ( 2 {g}_{o} + 2 {g}_{e} ) & if K = 0 \\ mN - ( 2 {g}_{o} + 2 {g}_{e} ) - 4 {Kg}_{e} & otherwise\end{matrix} .$$

where m is match reward, gₒ is gap opening penalty, gₑ is gap extension penalty, N is read length and K is size of the banded portion of the matrix.

In the second thresholding check, the high score is compared to the second score threshold, S2. If the high score is greater than the second score threshold, then variant calling is performed at 54 using the banded portion of the matrix.

Going back to the example embodiment, the second score threshold S2 (circled cell) is the largest possible score outside the band as seen in FIG. 6B. Passing S2 (the high score>S2) means the narrow-band score is guaranteed to be larger than any score within the region outside of the band as seen in FIG. 6B. Therefore, there cannot be a score outside the band that is larger than the narrow-band score, which means the score is optimal. With K=20, the passing rate of S2 is about 70%.

For those 30% of the extensions that fail the second thresholding check, a further mechanism can be used to reduce the number of extensions that need to be rerun. It can be described as an edit-distance check. The basic idea is to start from the first threshold score S1, and perform an extra alignment using an edit-distance machine, starting from the position corresponding to the cell of S1. In FIG. 7, the extra alignment is performed over the extra region noted by the highlighted box in lower left of the matrix. Since S1 is the best possible score at that cell and an edit-distance machine does not penalize gap openings, the alignment score (S-ed) would be an upper bound for the optimal score outside the band but within the extra region as seen in FIG. 7.

The precondition is that the narrow-band or high score (S-nb) passes S1 but fails S2, or S1<S-nb<=S2. If S-nb<=S1, the narrow-band score is fairly low, and should simply rerun for another portion of the matrix (e.g., the entire matrix); if S-nb>S2, it passes the thresholding. Only when S-nb is in between the two thresholds does it need to be examined against the edit-distance check. If S-ed is greater than or equal to S-nb, there is no guarantee that S-nb is optimal, and a rerun would be needed. But if S-ed<S-nb, the narrow-band score is optimal, as shown below.

Assume S-ed<S-nb and S-nb is not optimal, then there must exist a score S* outside the band and S*>S-nb. Since S-nb passes S1, S-nb is larger than any score in the highlighted area of FIG. 6A, then S* must be obtained somewhere outside the band in the extra region in FIG. 7. As discuss before, the edit-distance alignment score, S-ed, is an upper bound for this region, then S-ed>=S*. It follows that the relations are: S-ed>=S*, S*>S-nb, and from assumption, S-nb>S-ed, it reaches a contradiction. Therefore, the assumption is not true. When S-ed<S-nb, the narrow-band score is indeed optimal.

With continued reference to FIG. 5, an edit distance check involves computing an edit distance for the each cell in an extra region of the matrix. The extra region of the matrix is caused by the length of the reference string, L, being larger than the length of the read, N. In particular, the extra region is defined as a portion of the full matrix that corresponds to the portion of the read and the reference string following the cell of the first threshold. In the example of FIG. 7, the cell of the first threshold (circled cell) is at the 13^{th} row and the 8^{th} column. The extra region in this case is the portion of the matrix starting from the 14^{th} character of the reference string and the 9^{th} character of the read. The edit distance score (S-ed) is set as the highest edit distance from the cells in the extra region. As noted above, the condition for performing the edit distance check is that the high score is greater than the first score threshold and the high score is less than or equal to the second score threshold.

The narrow band high score is then compared to the edit distance score at 55. Variant calling is performed at 56 using the banded portion of the matrix if the edit distance score is less than (or equal to) the narrow band high score. Conversely, alignment scores are recomputed at 57 for another portion of the matrix (e.g., the entire matrix) using the sequence alignment algorithm if the edit distance score is greater than the narrow band high score.

For illustration purposes, the idea is implemented in software and ran on the data set ERR194147_10m_1.fastq. The output sam file matches perfectly with that of the original version of BWA-MEM. Here are the stats.

| | Passing S1 | Passing S1 | | |
|---|---|---|---|---|
| | but | and | | Total |
| Failing S1 | failing S-ed | passing S-ed | Passing S2 | extensions |
| 0 | 624974 | 18183723 | 47784201 | 66592898 |
| 0% | 0.94% | 27.31% | 71.76% | 100% |

The overall passing rate=71.76% (thresholding)+27.31% (edit-distance check)=99.07%. However, the time performance on software is not improved using these techniques. It is about 20% slower than the original BWA-MEM.

In sum, this variant of the alignment method uses a narrower band to perform the seed extension and two checks to ensure the narrow-band score is optimal. The first one is the thresholding check, and there are two thresholds, S1 and S2. If the narrow-band score does not pass S1, a rerun is needed; if the narrow-band score passes S2, it is guaranteed to be optimal. When the narrow-band score lies between the two thresholds, an edit-distance check is used. If the narrow-band score passes edit-distance check, it is guaranteed to be optimal, otherwise a rerun is needed. Overall, less than 1% of all extensions need to be rerun with a full band.

The techniques described herein may be implemented by one or more computer programs executed by one or more processors. The computer programs include processor-executable instructions that are stored on a non-transitory tangible computer readable medium. The computer programs may also include stored data. Non-limiting examples of the non-transitory tangible computer readable medium are nonvolatile memory, magnetic storage, and optical storage.

Some portions of the above description present the techniques described herein in terms of algorithms and symbolic representations of operations on information. These algorithmic descriptions and representations are the means used by those skilled in the data processing arts to most effectively convey the substance of their work to others skilled in the art. These operations, while described functionally or logically, are understood to be implemented by computer programs. Furthermore, it has also proven convenient at times to refer to these arrangements of operations as modules or by functional names, without loss of generality.

Unless specifically stated otherwise as apparent from the above discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices.

Certain aspects of the described techniques include process steps and instructions described herein in the form of an algorithm. It should be noted that the described process steps and instructions could be embodied in software, firmware or hardware, and when embodied in software, could be downloaded to reside on and be operated from different platforms used by real time network operating systems.

The present disclosure also relates to an apparatus for performing the operations herein. This apparatus may be specially constructed for the required purposes, or it may comprise a computer selectively activated or reconfigured by a computer program stored on a computer readable medium that can be accessed by the computer. Such a computer program may be stored in a tangible computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

The algorithms and operations presented herein are not inherently related to any particular computer or other apparatus. Various systems may also be used with programs in accordance with the teachings herein, or it may prove convenient to construct more specialized apparatuses to perform the required method steps. The required structure for a variety of these systems will be apparent to those of skill in the art, along with equivalent variations. In addition, the present disclosure is not described with reference to any particular programming language. It is appreciated that a variety of programming languages may be used to implement the teachings of the present disclosure as described herein.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A computer-implemented method for aligning an unknown string with a reference string, comprising:
receiving, by a computer processor, a banded portion of a matrix from a sequence alignment algorithm, where each column in the matrix corresponds to a character in the unknown string, each row in the matrix corresponds to a character in the reference string, and each cell in the matrix represents an alignment score between a portion of the unknown string and a portion of the reference string, such that a value of a given cell is the alignment score between characters in the unknown string that precede and include the given cell and the characters in the reference string that precede and include the given cell;
calculating, by the computer processor, a score threshold for the banded portion of the matrix, where value of the score threshold is calculated as a function of a scoring method used by the sequence alignment algorithm;
identifying, by the computer processor, a high score amongst the cells in the banded portion of the matrix;
comparing, by the computer processor, the high score to the score threshold; and
computing, by the computer processor, alignment scores for entire matrix using the sequence alignment algorithm in response to a determination that the high score is less than or equal to the score threshold.

2. The method of claim 1 further comprises performing variant calling using the banded portion of the matrix, where variant calling is performed in response to a determination that the high score is greater than to the score threshold.

3. The method of claim 1 further comprises calculating the score threshold as a function of length of the unknown string, the scoring method used by the sequence alignment algorithm, and size of the banded portion of the matrix.

4. The method of claim 1 wherein the scoring method is further defined as an edit distance method.

5. The method of claim 1 wherein the scoring method is further defined as affine gap penalty method.

6. The method of claim 5 further comprises calculating the score threshold in accordance with
$$scor {e}_{threshold} = \{ \begin{matrix}mN - ( m + {g}_{o} + {g}_{e} ) & if K = 0 \\ mN - ( m + {g}_{o} + {g}_{e} ) - 2 K ( {g}_{e} + m ) & otherwise\end{matrix} ,$$
where m is match reward, gₒ is gap opening penalty, gₑ is gap extension penalty, N is string length and K is size parameter of the banded portion of the matrix.

7. The method of claim 1 wherein the sequence alignment algorithm is at least one of Smith-Waterman algorithm or Needleman-Wunsch algorithm.

8. A method for aligning a read with a reference substring of a genome sequence, comprising:
receiving a first banded portion of a matrix from a sequence alignment algorithm, where each column in the matrix corresponds to a character in the read, each row in the matrix corresponds to a character in the reference substring, and each cell in the matrix represents an alignment score between a portion of the read and a portion of the reference substring, such that a value of a given cell is the alignment score between characters in the read that precede and include the given cell and the characters in the reference substring that precede and include the given cell;
calculating, by the computer processor, a score threshold for the banded portion of the matrix, where value of the score threshold is calculated as a function of a scoring method used by the sequence alignment algorithm;
identifying, by the computer processor, a high score amongst the cells in the banded portion of the matrix;
comparing, by the computer processor, the high score to the score threshold; and
computing, by the computer processor, alignment scores for a second portion of the matrix using the sequence alignment algorithm, where the second portion of the matrix is larger is size than the first banded portion of the matrix and the alignment scores are computed in response to a determination that the high score is less than or equal to the score threshold; and
performing, by the computer processor, variant calling using the banded portion of the matrix in response to a determination that the high score is greater than to the score threshold.

9. The method of claim 8 wherein the second portion of the matrix is another banded portion of the matrix provided by the sequence alignment algorithm.

10. The method of claim 8 wherein the second portion of the matrix is entire matrix provided by the sequence alignment algorithm.

11. The method of claim 8 further comprises calculating the score threshold as a function of length of the read, the scoring method used by the sequence alignment algorithm, and size of the banded portion of the matrix.

12. The method of claim 8 wherein the scoring method is further defined as affine gap penalty method.

13. The method of claim 12 further comprises calculating the score threshold in accordance with
$$scor {e}_{threshold} = \{ \begin{matrix}mN - ( m + {g}_{o} + {g}_{e} ) & if K = 0 \\ mN - ( m + {g}_{o} + {g}_{e} ) - 2 K ( {g}_{e} + m ) & otherwise\end{matrix} ,$$
where m is match reward, gₒ is gap opening penalty, gₑ is gap extension penalty, N is string length and K is size parameter of the banded portion of the matrix.

14. The method of claim 8 wherein the sequence alignment algorithm is at least one of Smith-Waterman algorithm or Needleman-Wunsch algorithm.

15. A computer-implemented method for aligning a read with a reference substring of a genome sequence, comprising:
receiving a first banded portion of a matrix from a sequence alignment algorithm, where each column in the matrix corresponds to a character in the read, each row in the matrix corresponds to a character in the reference substring, and each cell in the matrix represents an alignment score between a portion of the read and a portion of the reference substring, such that a value of a given cell is the alignment score between characters in the read that precede and include the given cell and the characters in the reference substring that precede and include the given cell;
identifying, by the computer processor, a high score amongst the cells in the banded portion of the matrix;
calculating, by the computer processor, a first score threshold for the first banded portion of the matrix, where value of the first score threshold is calculated as a function of a scoring method used by the sequence alignment algorithm;
comparing, by the computer processor, the high score to the first score threshold;
calculating, by the computer processor, a second score threshold for the banded portion of the matrix, where value of the second score threshold is larger than the first score threshold and is calculated as a function of a scoring method used by the sequence alignment algorithm;
comparing, by the computer processor, the high score to the second score threshold;
performing, by the computer processor, variant calling using the banded portion of the matrix in response to a determination that the high score is greater than to the first score threshold and the high score is greater than the second score threshold.

16. The method of claim 15 further comprises computing, by the computer processor, alignment scores for a second portion of the matrix using the sequence alignment algorithm, where the second portion of the matrix is larger is size than the first banded portion of the matrix and the alignment scores are computed in response to a determination that the high score is less than or equal to the score threshold.

17. The method of claim 16 further comprises
computing, by the computer processor, an edit distance score for an extra region of the matrix, where the edit distance score is computed in response to a determination that the high score is less than or equal to the second score threshold and the extra region is comprised of cells which follow a cell corresponding to the first threshold score;
comparing, by the computer processor, the high score to the edit distance score; and
performing, by the computer processor, variant calling using the banded portion of the matrix in response to a determination that the high score is greater than the edit distance score.

18. The method of claim 17 further comprises computing, by the computer processor, alignment scores for a second portion of the matrix using the sequence alignment algorithm, where the second portion of the matrix is larger is size than the first banded portion of the matrix and the alignment scores are computed in response to a determination that the edit distance score is less than or equal to the high score.

19. The method of claim 15 further comprises calculating the second score threshold in accordance with
$$scor {e}_{threshold} = \{ \begin{matrix}mN - ( {g}_{o} + {g}_{e} ) & if K = 0 \\ mN - ( {g}_{o} + {g}_{e} ) - 2 {Kg}_{e} & otherwise\end{matrix} ,$$
where m is match reward, gₒ is gap opening penalty, gₑ is gap extension penalty, N is length of read and K is size parameter of the banded portion of the matrix.
